(19) United States
(12) Patent Application Publication
(10) Pub. No.: US 2020/0323813 A1
(43) Pub. Date: Oct. 15, 2020

(54) TREATMENT OF VIRAL HEMORRHAGIC FEVERS WITH COX-2 SELECTIVE NON-STEROIDAL ANTI-INFLAMMATORY DRUGS

(71) Applicant: Tremeau Pharmaceuticals, Inc., Concord, MA (US)

(72) Inventors: Bradford C. SIPPY, Acton, MA (US); Travis E. HELM, Acton, MA (US)

(21) Appl. No.: 16/843,497

(22) Filed: Apr. 8, 2020

Related U.S. Application Data
(60) Provisional application No. 62/831,407, filed on Apr. 9, 2019.

(51) Int. Cl. A61K 31/341; A61K 9/00; A61P 31/14; A61P 29/00; A61K 31/415; A61K 31/42; A61K 31/196; A61K 31/167

(52) U.S. Cl. CPC A61K 31/341; A61K 9/0053; A61P 31/14; A61K 31/167; A61K 31/415; A61K 31/42; A61K 31/196; A61P 29/00

(57) ABSTRACT

The present disclosure relates to a method of treating symptoms of fever, pain, and inflammation in patients infected with a Viral Hemorrhagic Fever, the method comprising administering a COX-2 selective Non-Steroidal Anti-Inflammatory Drug (NSAID) at a sufficient dose to reduce both the symptoms and viral level of the infection, and matching the dose of a COX-2 selective NSAID to the time course of the Viral Hemorrhagic Fever to provide maximum anti-pyretic effect during the febrile phase of the infection and retain a lower maintenance dose during the remainder of the infection.

This application claims the benefit of and priority to U.S. Provisional Patent Application No. 62/831,407 filed on Apr. 9, 2019, the content of which is hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Description of Related Art—Viral Hemorrhagic Fevers, Clinical Manifestations, and Management Thereof

Viral Hemorrhagic Fevers

The Viral Hemorrhagic Fevers (VHFs) are a diverse group of illnesses caused by RNA viruses derived from four viral families linked by a clinical syndrome. These four families include the Arenaviridae, Bunyaviridae, Filoviridae, and Flaviviridae. The Arenaviridae include etiologic agents of Argentine, Bolivian and Venezuelan Hemorrhagic Fevers and Lassa Fever. The Bunyaviridae include members of the Hantavirus genus, the Congo-Crimean Hemorrhagic Fever virus from the Nairovirus genus, and the Rift Valley Fever virus from the Phlebovirus genus. The Filoviridae include Ebola and Marburg viruses. The Flaviviridae include Dengue and Yellow Fever viruses. These viruses are spread to humans in a variety of ways including, but not limited to, direct contact (i.e., droplet transmission from an infected human), indirect contact (i.e., animal to human and insect vector to human) or transmission via entry of a respiratory portal. A summary further describing the four key VHFs is provided below.

Arenaviridae:

Argentine Hemorrhagic Fever (AHF), caused by the Junin virus, was discovered in 1955, following reports of AHF symptoms found in corn harvesters. Today, nearly 300 to 600 cases per year are reported in regions of the Argentine pampas. The Bolivian, Brazilian and Venezuelan Hemorrhagic Fevers are caused by related Machupo, Guanarito and Sabia viruses, with the Lassa virus being a common cause for disease in West Africa. These viruses are transmitted from their rodent reservoirs to humans by the inhalation of dust particles contaminated with rodent excreta.

Bunyaviridae:

Congo-Crimean Hemorrhagic Fever (CCHF) is primarily a tick-borne disease found in Crimea and in parts of Africa, Europe and Asia. However, CCHF can also be spread by contact with infected animals and in healthcare facilities treating CCHF infected patients. Rift Valley Fever (RVF) is a mosquito-borne disease in Africa. The hantaviruses are rodent-borne viruses with a wide geographic distribution. Hantaan, and closely related viruses, cause hemorrhagic fever with renal syndrome (HFRS, also known as Korean Hemorrhagic Fever or Epidemic Hemorrhagic Fever) and is reportedly the most common disease due to the hantaviruses. Prior to World War II, Hantaan was described as being found in Manchuria along the Amur River, infected among United Nations troops during the Korean conflict, and subsequently in Japan, China and in the Russian Far East. Severe disease also occurs in some Balkan states, including Bosnia, Serbia and Greece. Nephropathia epidemica is a milder disease found in Scandinavia and in other parts of Europe, and is caused by strains carried by bank voles. In addition, newly described hantaviruses cause Hantavirus Pulmonary Syndrome (HPS) in the Americas. The hantaviruses are also transmitted to humans by the inhalation of dust particles contaminated with rodent excreta.

Filoviridae:

Ebola Hemorrhagic Fever was first recognized in 1976, in the western equatorial province of Sudan and the nearby region of Zaire. A second outbreak occurred in Sudan in 1979, and larger outbreak in 1995, comprising of at least 316 cases, which developed in Kikwit, Zaire from a single index case. Subsequent epidemics have also occurred in Gabon, the Ivory Coast, Uganda and the Republic of Congo. There are five species of Ebola: Zaire, Sudan, Ivory Coast, Reston and Bundibugyo. These African strains can cause severe disease and even death, with case fatality rates that vary by viral species (i.e., Bundibugyo ˜35%, Sudan 40%-50%, Zaire 80%-90%). At this time, it is not known why this disease appears infrequently. A related virus, the Ebola Reston, was isolated from monkeys in 1989, and these monkeys subsequently developed hemorrhagic fever. While subclinical infections occurred among exposed animal handlers, Ebola Reston has not been identified as a human pathogen despite being shown to cause disease in pigs and non-human primates. Marburg epidemics have occurred on eight separate occasions: six times in Africa and twice in Europe. The first recognized outbreak occurred in Marburg, Germany and in Yugoslavia among people exposed to African green monkeys; which resulted in 31 contracted cases and seven deaths. Overall, case fatality rates of the outbreaks in Marburg have varied from 21% to nearly 90%. While there is only one specie of the Marburg virus, there exists several strains. Filoviruses can be spread from human to human by direct contact with infected blood, secretions, organs, or semen. Ebola Reston apparently spread from monkey to monkey, as well as from monkey to human by the respiratory route. While the natural reservoirs of the filoviruses are unknown, recent evidence strongly implicates bats as either the reservoir or as the intermediate host.

Flaviviridae:

Yellow and Dengue Fever are two mosquito-borne hemorrhagic fevers of the Flaviviridae family of viruses that have great importance in the history of military campaigns and military medicine, as well as in port cities engaging in commerce with the tropics, such as, for example, in New Orleans. Tick-borne hemorrhagic flaviviruses include the agents of Kyanasur Forest disease in India, and Omsk Hemorrhagic Fever in Siberia. The Zika virus in a non-hemorrhaging member of the Flaviviridae family.

Specifically, Dengue Fever is a mosquito-borne tropical disease caused by the Dengue Virus, and is a member of the Flaviviridae family of viruses. Symptoms usually last up to 14 days and may include a sudden high-grade fever, headache, vomiting, rash, and extreme pain and stiffness of the joints. Though generally yielding a lower mortality rate than some other VHFs, the extreme pain and joint inflammation associated with Dengue Fever has earned it the pseudonym “Breakbone Fever.” Symptoms in severe cases may include internal and external bleeding (i.e., Dengue Hemorrhagic Fever). An estimated 96 million clinical cases of Dengue Fever occur annually worldwide and it is a leading cause of childhood death in Asia and Latin America. As Dengue Fever has been a highly prevalent and well-known disease to modern medicine for many decades, significant detail regarding the time course of the disease, symptom manifestation, and relationship between viral load and fever level to the severity of the disease has been elucidated. Following an estimated 4-10 day incubation period after infection, the time course of Dengue Fever follows three distinct phases: the Febrile Phase, the Critical Phase, and the Recovery Phase.

During the Febrile Phase, patients typically will rapidly develop a high-grade fever. This acute febrile phase usually lasts between about 2-7 days and is often accompanied by facial flushing, skin erythema, generalized body ache, myalgia, severe arthralgia and headache. Some patients may also experience a sore throat, injected pharynx and conjunctival injection. Anorexia, nausea and vomiting are also common. Because of this, it can be difficult to clinically distinguish Dengue from other hemorrhagic fevers in the early febrile phase. However, a positive tourniquet test, indicating vascular fragility, in this phase increases the probability of differential Dengue diagnosis. In addition, these clinical features are indistinguishable between severe and non-severe Dengue cases. Mild hemorrhagic manifestations like petechiae and mucosal membrane bleeding (e.g., nose and gums) may also be seen. Even more, extremely heavy vaginal bleeding (particularly in women of childbearing age) and gastrointestinal bleeding may occur during this phase, but is not common, and the liver may often become enlarged and tender after a few days of fever. In addition to primary manifestations of Dengue, secondary disease manifestations related to high fever (e.g., febrile seizures, especially in children) can occur at this phase.

The Critical Phase usually commences on day 3-7 of the illness after the internal temperature of the patient drops to 37.5-38 degrees C. or less and remains below this level. At that time, an increase in capillary permeability in parallel with increasing hematocrit levels may occur. If it occurs, the period of clinically significant plasma leakage initiates at this phase and will usually last between about 24-48 hours. Though uncommon, shock may occur when a critical volume of plasma is lost through leakage. With prolonged shock, the consequent organ hypoperfusion results in progressive organ impairment, metabolic acidosis and disseminated intravascular coagulation. This in turn leads to severe hemorrhage causing the hematocrit to decrease in severe shock. In addition, severe organ impairment, such as, for example, severe hepatitis, encephalitis or myocarditis and/or severe bleeding may develop without obvious plasma leakage or shock.

Patients with severe Dengue may have coagulation abnormalities, but these are usually not sufficient enough to cause major bleeding. When major bleeding does occur, it is almost always associated with profound shock because this, in combination with thrombocytopenia, hypoxia and acidosis, can lead to multiple organ failure and advanced disseminated intravascular coagulation. Massive bleeding may occur without prolonged shock in instances when acetylsalicylic acid (aspirin), ibuprofen or corticosteroids have been taken.

The Recovery Phase is characterized by a gradual reabsorption of extravascular compartment fluid over 2-3 days after the Critical Phase ends. At that time, the general well-being of the patient improves, appetite returns, gastrointestinal symptoms abate, hemodynamic status stabilizes, and diuresis ensues. Some patients may have a rash of what is known as the “isles of white in the sea of red,” while others may experience generalized pruritus. Bradycardia and electrocardiographic changes are also common during this stage.

Existing paradigms express that the risk of progression to more severe forms of Dengue Fever is correlated with fever severity (i.e., fevers greater than 38 degrees C. associated with risk of severe Dengue) and viral load (i.e., viral loads higher during days 1-2 for severe versus normal Dengue) during the Febrile Phase. In addition, existing paradigms express that the risk of some secondary disease manifestations (e.g., febrile seizures) is correlated to fever severity during the Febrile Phase.

The Zika virus (ZIKV) is a member of the Flaviviridae family of viruses and is related to Dengue Fever and Yellow Fever. Its name comes from the Zika Forest of Uganda, where the virus was first isolated in 1947. The infection, also known as Zika Fever or Zika virus disease, often causes no or only mild symptoms similar to a very mild form of Dengue Fever. While Zika, in and of itself, does not manifest into a hemorrhagic fever because it is endemic in similar areas to Dengue and its early clinical onset is similar, treatment constraints applied to Dengue, such as avoidance of NSAIDs, are also applied to Zika.

### SUMMARY OF THE INVENTION

In certain aspects, the invention provides a method for treating one or more symptoms in a subject having Viral Hemorrhagic Fever, the method comprising administering a therapeutically effective amount of a COX-2 selective Non-Steroidal Anti-Inflammatory Drug (NSAID) or a pharmaceutically acceptable salt thereof.

In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the Viral Hemorrhagic Fever is caused by one or more RNA viruses derived from a family of viruses selected from Arenaviridae, Bunyaviridae, Filoviridae, Flaviviridae, and any combinations thereof. In some embodiments, the Viral Hemorrhagic Fever is caused by one or more RNA viruses from the Flaviviridae family of viruses. In some embodiments, the Viral Hemorrhagic Fever is caused by a Dengue Virus.

In some embodiments, the therapeutically effective amount of the COX-2 selective NSAID causes a reduction in viral load. In some embodiments, the COX-2 selective NSAID is administered orally. In some embodiments, the COX-2 selective NSAID is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the COX-2 selective NSAID is provided in a quantity sufficient for a single course of therapy. In some embodiments, the therapeutically effective amount of the COX-2 selective NSAID is provided in a single course of therapy package.

In some embodiments, the therapeutically effective amount of the COX-2 selective NSAID is at a dose matched to the time course of the Viral Hemorrhagic Fever. In some embodiments, the dose provides maximum anti-pyretic effect during the febrile phase of an infection. In some embodiments, the method further comprises administering a lower maintenance dose during the critical phase and/or recovery phase of the infection compared to the dose matched to the time course of the Viral Hemorrhagic Fever.

In some embodiments, the COX-2 selective NSAID is administered as a loading dose during a febrile stage of a viral infection, and a lower maintenance dose during the critical phase and/or recovery phase of the infection. In some embodiments, the COX-2 selective NSAID is administered at a dose that does not affect platelet aggregation or bleeding time.

In some embodiments, the COX-2 selective NSAID is selected from celecoxib, rofecoxib, valdecoxib, parecoxib, lumiracoxib, and any combinations thereof. In some embodiments, the COX-2 selective NSAID is rofecoxib.

In some embodiments, the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg. In some embodiments, the rofecoxib is administered at a dose of about 12.5 mg/day, about 15 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In some embodiments, the COX-2 selective NSAID is administered in combination with acetaminophen. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 15 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with a virus capable of causing Viral Hemorrhagic Fever, the method comprising: assessing the subject for one or more symptoms of Viral Hemorrhagic Fever, and if one or more symptoms of Viral Hemorrhagic Fever is present, administering a therapeutically effective amount of a COX-2 selective Non-Steroidal Anti-Inflammatory Drug (NSAID) or a pharmaceutically acceptable salt thereof to the subject.

In some embodiments, the method further comprises reassessing symptoms following the administration of the therapeutically effective amount of the COX-2 selective NSAID; and adjusting treatment according to the reassessed symptoms, wherein the adjusting comprises administering further COX-2 selective NSAID if the reassessed symptoms are worse, not improved or improved but not gone compared to the assessed symptoms and terminating administering COX-2 selective NSAID if the reassessed symptoms are gone compared to the assed symptoms. In some embodiments, the method further comprises identifying the virus capable of causing Viral Hemorrhagic Fever in the infected subject.

In some embodiments, the therapeutically effective amount of the COX-2 selective NSAID comprises a dose and duration of treatment sufficient to suppress viral replication of the identified viral pathogen or decrease viral load. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with a virus capable of causing Viral Hemorrhagic Fever, the method comprising: administering to the subject a COX-2 selective Non-Steroidal Anti-Inflammatory Drug (NSAID) or a pharmaceutically acceptable salt thereof at a sufficient dose to cause a reduction in viral load of the virus.

In some embodiments, the Viral Hemorrhagic Fever is caused by one or more RNA viruses derived from a family of viruses selected from Arenaviridae, Bunyaviridae, Filoviridae, Flaviviridae, and any combinations thereof. In some embodiments, the Viral Hemorrhagic Fever is caused by one or more RNA viruses from the Flaviviridae family of viruses. In some embodiments, the Viral Hemorrhagic Fever is caused by a Dengue Virus.

In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day, followed by a maintenance dose of about 12.5 mg/day, about 20 mg/day, about 25 mg/day, about 30 mg/day, about 40 mg/day, or about 50 mg/day.

In certain aspects, the invention provides a method for treating one or more symptoms in a subject having symptoms of Zika Virus infection, the method comprising: administering to the subject a COX-2 selective Non-Steroidal Anti-Inflammatory Drug (NSAID) or a pharmaceutically acceptable salt thereof such that the subject experiences a reduction in the one or more symptoms.

In some embodiments, the COX-2 selective NSAID is administered orally. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with Zika Virus, the method comprising: administering to the subject a COX-2 selective NSAID or a pharmaceutically acceptable salt thereof at a sufficient dose such that the subject experiences a reduction in viral load of the Zika virus.

In some embodiments, the COX-2 selective NSAID is administered orally. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

### BRIEF DESCRIPTION OF FIGURES

FIG. 1 illustrates an embodiment of the disclosure for a method of treating a patient infected with a virus capable of causing Viral Hemorrhagic Fever by administering a COX-2 selective NSAID.

### DETAILED DESCRIPTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein, “rofecoxib” refers to the active ingredient 4-[4-(methylsulfonyl)phenyl]-3-phenyl-2(5H)-furanone or a pharmaceutically acceptable salt or solvate thereof.

As used herein, “administered in combination” means the administration of two agents (e.g., concomitantly or sequentially) in any manner in which the pharmacological effects of both are manifest in the subject at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals. The term “mammal” includes, but is not limited to, a human, mouse, rat, guinea pig, dog, cat, horse, cow, pig, or non-human primate, such as a monkey, chimpanzee, baboon or rhesus. In one embodiment, the mammal is a human.

As used herein, the term “patient” refers to a human or animal.

In certain aspects, the invention provides a method for treating one or more symptoms in a subject having Viral Hemorrhagic Fever, the method comprising administering a therapeutically effective amount of a COX-2 selective Non-Steroidal Anti-Inflammatory Drug (NSAID) or a pharmaceutically acceptable salt thereof.

In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the Viral Hemorrhagic Fever is caused by one or more RNA viruses derived from a family of viruses selected from Arenaviridae, Bunyaviridae, Filoviridae, Flaviviridae, and any combinations thereof. In some embodiments, the Viral Hemorrhagic Fever is caused by one or more RNA viruses from the Flaviviridae family of viruses. In some embodiments, the Viral Hemorrhagic Fever is caused by a Dengue Virus.

In some embodiments, the therapeutically effective amount of the COX-2 selective NSAID causes a reduction in viral load. In some embodiments, the COX-2 selective NSAID is administered orally. In some embodiments, the COX-2 selective NSAID is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the COX-2 selective NSAID is provided in a quantity sufficient for a single course of therapy. In some embodiments, the therapeutically effective amount of the COX-2 selective NSAID is provided in a single course of therapy package.

In some embodiments, the therapeutically effective amount of the COX-2 selective NSAID is at a dose matched to the time course of the Viral Hemorrhagic Fever. In some embodiments, the dose provides maximum anti-pyretic effect during the febrile phase of an infection. In some embodiments, the method further comprises administering a lower maintenance dose during the critical phase and/or recovery phase of the infection compared to the dose matched to the time course of the Viral Hemorrhagic Fever.

In some embodiments, the COX-2 selective NSAID is administered as a loading dose during a febrile stage of a viral infection, and a lower maintenance dose during the critical phase and/or recovery phase of the infection. In some embodiments, the COX-2 selective NSAID is administered at a dose that does not affect platelet aggregation or bleeding time.

In some embodiments, the COX-2 selective NSAID is selected from celecoxib, rofecoxib, valdecoxib, parecoxib, lumiracoxib, and any combinations thereof. In some embodiments, the COX-2 selective NSAID is rofecoxib.

In some embodiments, the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg. In some embodiments, the rofecoxib is administered at a dose of about 12.5 mg/day, about 15 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In some embodiments, the COX-2 selective NSAID is administered in combination with acetaminophen. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 15 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with a virus capable of causing Viral Hemorrhagic Fever, the method comprising: assessing the subject for one or more symptoms of Viral Hemorrhagic Fever, and if one or more symptoms of Viral Hemorrhagic Fever is present, administering a therapeutically effective amount of a COX-2 selective Non-Steroidal Anti-Inflammatory Drug (NSAID) or a pharmaceutically acceptable salt thereof to the subject.

In some embodiments, the method further comprises reassessing symptoms following the administration of the therapeutically effective amount of the COX-2 selective NSAID; and adjusting treatment according to the reassessed symptoms, wherein the adjusting comprises administering further COX-2 selective NSAID if the reassessed symptoms are worse, not improved or improved but not gone compared to the assessed symptoms and terminating administering COX-2 selective NSAID if the reassessed symptoms are gone compared to the assed symptoms. In some embodiments, the method further comprises identifying the virus capable of causing Viral Hemorrhagic Fever in the infected subject.

In some embodiments, the therapeutically effective amount of the COX-2 selective NSAID comprises a dose and duration of treatment sufficient to suppress viral replication of the identified viral pathogen or decrease viral load. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with a virus capable of causing Viral Hemorrhagic Fever, the method comprising: administering to the subject a COX-2 selective Non-Steroidal Anti-Inflammatory Drug (NSAID) or a pharmaceutically acceptable salt thereof at a sufficient dose to cause a reduction in viral load of the virus.

In some embodiments, the Viral Hemorrhagic Fever is caused by one or more RNA viruses derived from a family of viruses selected from Arenaviridae, Bunyaviridae, Filoviridae, Flaviviridae, and any combinations thereof. In some embodiments, the Viral Hemorrhagic Fever is caused by one or more RNA viruses from the Flaviviridae family of viruses. In some embodiments, the Viral Hemorrhagic Fever is caused by a Dengue Virus.

In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day, followed by a maintenance dose of about 12.5 mg/day, about 20 mg/day, about 25 mg/day, about 30 mg/day, about 40 mg/day, or about 50 mg/day.

In certain aspects, the invention provides a method for treating one or more symptoms in a subject having symptoms of Zika Virus infection, the method comprising: administering to the subject a COX-2 selective Non-Steroidal Anti-Inflammatory Drug (NSAID) or a pharmaceutically acceptable salt thereof such that the subject experiences a reduction in the one or more symptoms.

In some embodiments, the COX-2 selective NSAID is administered orally. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

In certain aspects, the invention provides a method for treating a subject infected with Zika Virus, the method comprising: administering to the subject a COX-2 selective NSAID or a pharmaceutically acceptable salt thereof at a sufficient dose such that the subject experiences a reduction in viral load of the Zika virus.

In some embodiments, the COX-2 selective NSAID is administered orally. In some embodiments, the COX-2 selective NSAID is selected from celecoxib, rofecoxib, valdecoxib, parecoxib, lumiracoxib, and combinations thereof. In some embodiments, the COX-2 selective NSAID is rofecoxib.

In some embodiments, the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the rofecoxib is administered at a dose of about 12.5 mg/day, about 20 mg/day, about 25 mg/day, about 30 mg/day, about 40 mg/day, or about 50 mg/day.

In some embodiments, the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day. In some embodiments, the method further comprises administering a maintenance dose of rofecoxib of about 12.5 mg/day, about 20 mg/day, about 25 mg/day, about 30 mg/day, about 40 mg/day, or about 50 mg/day. In some embodiments, the maintenance dose of rofecoxib is administered for up to 3 days, up to 5 days, up to 7 days, up to 10 days, or up to 14 days.

Clinical Description of Viral Hemorrhagic Fevers

Though diverse in the underlying pathogens, common symptoms of VHFs include fever, myalgia, arthralgia, nausea, vomiting, and prostration. Physical examination may reveal only conjunctival injection, mild hypotension, flushing and petechial hemorrhages. Severe VHF typically evolves to shock and generalized mucous membrane hemorrhage and often is accompanied by evidence of pulmonary hematopoietic and neurologic involvement. Renal insufficiency is proportional to cardiovascular compromise, except in HFRS, which features renal failure as an integral part of the disease process.

The clinical syndrome that these viruses may cause is VHF, however, this syndrome is variable in its presentation. Bleeding may be an uncommon feature and not very impressive when it occurs (as in mild forms of Dengue Fever or Rift Valley Fever) or it may present as a copious life-threatening hemorrhage, as in Crimean Congo Hemorrhagic Fever. The progression to a septic shock-like state may be due to a combination of increases in vascular permeability, vasodilation, decreased myocardial function and fluid loss.

VHF should be suspected in any patient presenting with a severe febrile illness and evidence of vascular involvement (postural hypotension, petechiae, easy bleeding, flushing of face and chest, non-dependent edema), who has traveled to an area where the virus is known to occur, or where infectious disease surveillance suggests an outbreak. Symptoms and signs suggesting additional organ system involvement are common (e.g., headache, photophobia, pharyngitis, cough, nausea or vomiting, diarrhea, constipation, abdominal pain, hyperesthesia, dizziness, confusion, tremor), but usually do not dominate the clinical presentation. A positive tourniquet test has been particularly useful in Dengue Hemorrhagic Fever, but should be sought in other hemorrhagic fevers as well.

Not all infected patients develop VHF. There is both divergence and uncertainty about which host factors and viral strain characteristics might be responsible for the mechanisms of disease. For example, an immunopathogenic mechanism has been identified for Dengue Hemorrhagic Fever, which usually occurs among patients previously infected with a heterologous Dengue serotype. Antibodies directed against the previous strain enhances uptake of the Dengue Virus by circulating monocytes. These cells express viral antigens on their surfaces. Lysis of the infected monocytes by cytotoxic T-cell responses results in the release of pro-inflammatory cytokines, pro-coagulants and anticoagulants, which in turn results in vascular injury and permeability, complement activation and a systemic coagulopathy.

Diffuse or disseminated intravascular coagulation (DIC) has been implicated in Rift Valley, Marburg and Ebola Fevers, but in most VHFs the etiology of the coagulopathy is multifactorial (e.g., hepatic damage, consumptive coagulopathy and primary marrow injury to megakaryocytes).

Treatment of VHFs

There are currently no curative treatments for VHFs, though some antiviral therapies, such as, for example, ribavirin, have demonstrated some utility in treating several of these infections. Other preventative or curative treatments for VHFs are under investigation. The primary treatments for patients with VHFs are supportive care to address acute symptom manifestation and, depending on the severity of symptoms and underlying health of the infected patient, intensive supportive care as required.

General principles of supportive care apply to hemodynamic, hematologic, pulmonary and neurologic manifestations of VHF, regardless of the specific etiologic agent. These patients all require intensive care, but the effectiveness of supportive care leads to variable outcomes depending on etiology of disease (i.e., very good in Dengue Hemorrhagic Fever, but not very effective in Yellow Fever). Management of these diseases is similar to the management of septic shock.

Health care providers employing vigorous fluid resuscitation of hypotensive patients must be mindful of the propensity of some VHFs (e.g., HFRS) for pulmonary capillary leak. Pressor agents are frequently required to address hypotension. The use of intravascular devices and invasive hemodynamic monitoring must be carefully considered in the context of potential benefit versus the risk of hemorrhage. Restlessness, confusion, myalgia, and hyperesthesia should be managed by conservative measures, and the judicious use of sedatives and analgesics/anti-pyretic such as, for example, acetaminophen. Secondary infections may occur as with any patient undergoing intensive care utilizing invasive procedures and devices, such as intravenous lines and indwelling catheters.

The management of clinical bleeding should follow the same principles as for any patient with a systemic coagulopathy and assisted by coagulation studies. Intramuscular injections, aspirin or any NSAIDs (e.g., ibuprofen, celecoxib) and other anticoagulant drugs should be avoided.

Existing paradigms express that the over expression of cyclooxygenase-2 (COX-2) plays a role in the replication of several types of viruses, including Hepatitis C and Dengue, and it has been hypothesized that the inhibition of COX-2 via COX-2 inhibitors such as traditional NSAIDs or selective COX-2 inhibitors could serve as a treatment option in certain viral infections. However, due to the confounding risk of exacerbating gastritis and hemorrhage in patients infected with the Dengue Virus, use of NSAIDs and COX-2 selective NSAIDs (e.g., celecoxib) are not recommended to treat the symptoms of Dengue.

Description of Related Art—COX-2 Selective NSAIDs

Non-steroidal anti-inflammatory drugs (NSAIDs) are among the most widely used drugs in the world. They are chiefly used to treat inflammatory-related pain conditions, but their short-term use is limited because of the effects on bleeding time and platelet aggregation in situations with hemorrhagic risk, and their long-term use is limited by serious gastrointestinal side-effects. NSAIDs inhibit the two recognized forms of prostaglandin G/H synthase (also referred to as cyclo-oxygenase [COX]), namely COX-1 and COX-2. Since the analgesic, anti-pyretic, and anti-inflammatory effects of NSAIDs are mediated by inhibition of COX-2, and their platelet aggregation and gastrointestinal side effects mostly influenced by inhibition of COX-1, NSAIDs which selectively inhibit COX-2 theoretically reduce the risk of hemorrhagic effects and gastrointestinal toxicity compared with other NSAIDs while still providing analgesic, anti-pyretic, and anti-inflammatory effects.

On the premise of this theoretical difference, several COX-2 selective NSAIDs, such as rofecoxib and celecoxib, were developed in the 1990s. Early trials comparing selective COX-2 NSAIDs versus non-selective NSAIDs, such as naproxen and ibuprofen, seemed to confirm that the selective COX-2 NSAIDs at doses with similar analgesic efficacy had less gastrointestinal toxicity. In addition, COX-2 selective NSAIDs have demonstrated they do not affect platelet thromboxane production and do not impair platelet function, unlike non-selective NSAIDs. However, placebo-controlled trials have shown unequivocally that COX-2 selective NSAIDs are associated with an increased risk of atherothrombotic vascular events. Subsequent to these findings, several COX-2 selective NSAIDs were promptly removed from the market and/or non-approved. Analyses also showed that the CV effects of traditional NSAIDs and COX-2 selective NSAIDs were dose and duration dependent, with the highest risk being shown with chronic use of otherwise acute use doses.

Though later analyses have demonstrated that the cardiovascular (CV) effects of non-selective NSAIDs and COX-2 selective NSAIDs appear to be proportional to the magnitude of COX-2 inhibition and, therefore, equa-potent doses of non-selective NSAIDs appear to carry similar CV risks to COX-2 selective NSAIDs, the use of COX-2 selective NSAIDs in general has fallen out of favor and their use is not generally recommended.

Finally, current systems express non-selective NSAIDs and selective COX-2 NSAIDs as having known anti-pyretic (e.g., fever reducing) properties. Though a pronounced and potentially useful pharmacological effect, existing paradigms express that this property is a potential safety concern as, when used in the context of the treatment of acute or inflammatory pain, these products, in reducing fever, could mask the symptoms of an underlying and untreated infection.

Though all COX-2 selective NSAIDs share a common demonstration of little to no effect on bleeding time or anti-platelet inhibition at clinical doses used for pain, among the class there are wide differences on variables such as: selectivity for COX-2 isozyme, GI safety, half-live, onset of action, and dosing interval.

Problems in Related Art

The cluster of Viral Hemorrhagic Fevers (VHFs) and their related pseudo-similar diseases (e.g., Zika Virus), though diverse in their viral origins, all carry similar phenotypical symptoms and corresponding treatment related challenges. Each of the diseases manifest with rapid high-grade fever (pyrexia), pain, inflammation, nausea, vomiting, rash, and, except for Zika, varying degrees of potential hemorrhagic events. In addition, existing paradigms express that disease morbidity and outcomes are correlated to the initial severity of the fever and initial viral levels. Finally, existing paradigms express that COX-2 expression may play a role in the replication of some types of viruses, including replication of the Dengue Virus.

However, due to concerns with exacerbation of potential bleeding risk and gastritis, current paradigms express away from using known effective anti-pyretic and anti-inflammatory such as non-selective NSAIDs and COX-2 selective NSAIDs to address fever and inflammation associated with VHFs, and rather express the use of acetaminophen to address fever and pain. However, acetaminophen's properties (i.e., no anti-inflammatory properties, requires frequent and large doses, significant dose-related hepatic toxicities) limits its utility in this role.

In addition to these limitations specific to VHFs, current paradigms in general do not recommend using COX-2 selective NSAIDs due to concerns regarding cardiovascular safety. Though many existing paradigms express this as being an absolute risk, evidence indicates that CV risk with COX-2 selective NSAIDs is a dose and duration dependent effect.

### Non-Limiting Embodiments of the Disclosure

Though current paradigms do not recommend the use of non-selective NSAIDs and COX-2 selective NSAIDs in the treatment of VHFs, nor the use of COX-2 selective NSAIDs in the general population due to cardiovascular risks, the rationale for these treatment decisions is overly broad. An unobvious conclusion is that the clinical limitations of certain COX-2 selective NSAIDs are not necessarily applicable in patients suffering from an acute, short duration (often less than 14 days), high morbidity, potentially high mortality, and self-limiting condition, such as, for example, a VHF, and short-term use of COX-2 selective NSAIDs can provide significant benefits to patients suffering from VHFs, including Dengue Fever.

Specifically, a sudden, acute, short duration, high morbidity, high mortality, and self-limiting viral infection with fever, inflammation, nausea, and risk of hemorrhage as key clinical manifestations can be well addressed with certain COX-2 selective NSAIDs, but for which current paradigms do not recommend.

In addition, as fever severity in patients with VHFs is correlated with disease morbidity and possibly outcomes, an unobvious conclusion is that direct pharmacological intervention with a COX-2 selective NSAID with a potent anti-pyretic effect for a short duration can address fever in these patients in such a way to influence the outcome of the disease, while avoiding the toxicities associated with long-term use of these agents.

Finally, as some systems express that viral load of the Dengue Virus is correlated with disease severity and that COX-2 inhibition could play a role in Dengue viral replication, an unobvious conclusion is that direct pharmacological intervention with a potent COX-2 selective NSAID with a long half-life and sustained inhibitory effect for a short duration can suppress COX-2 replication in these patients, reduce disease severity, and minimize or avoid the toxicities associated with long-term use of COX-2 selective NSAIDs.

The present disclosure provides for a method of treating fever, pain, and inflammation in a patient infected with a virus capable of causing VHF comprising: administering (e.g., orally or parenterally) to the patient a COX-2 selective NSAID at a sufficient dose such that the patient experiences a reduction in the symptoms of fever, pain, and inflammation.

Further, the present disclosure provides for a method of matching the dose of a COX-2 selective NSAID to the time course of the VHF to provide maximum anti-pyretic effect during the febrile phase of the infection, and then stepping down to a lower maintenance dose during the remainder of the infection.

Further, the present disclosure provides for a method of treating a patient infected with a virus capable of causing Viral Hemorrhagic Fever comprising: administering to the patient a COX-2 selective NSAID at a sufficient dose to cause a reduction in the viral level or viral load of said virus. Viral load is also known as viral burden, viral titre or viral titer. Viral load is a numerical expression of the quantity of virus in a given volume. It is often expressed as particles per ml.

Finally, in order to further address the risk of chronic use of high doses of COX-2 selective NSAIDs, the present disclosure provides for a method of administering the COX-2 selective NSAID in a single course of therapy package, similar to unit of use packaging used for other short course treatments such as anti-biotics, so larger quantities of the intended dose are not easily available to a patient at any one time.

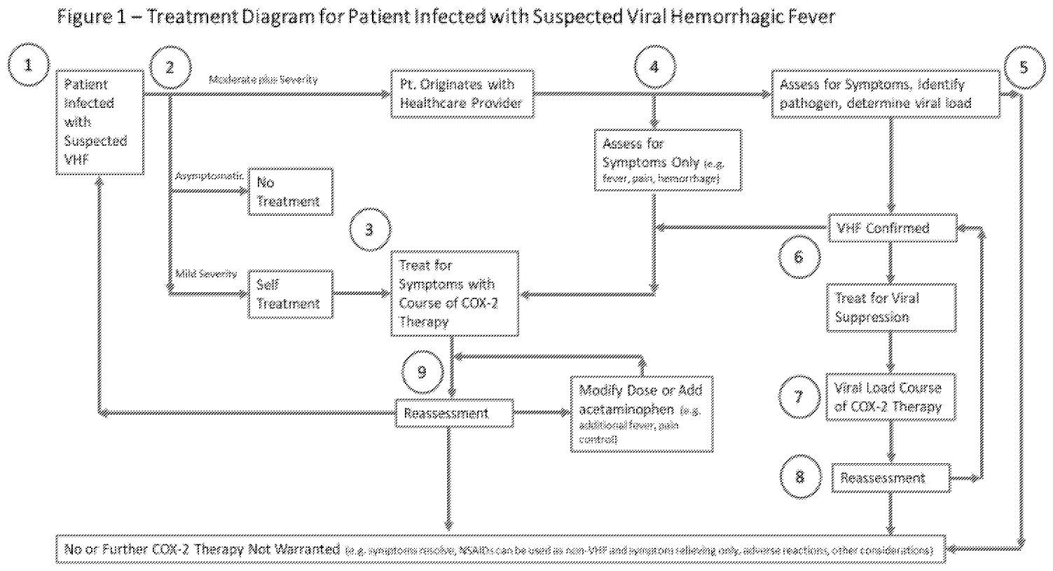

FIG. 1 illustrates an embodiment of the disclosure for a method of treating a patient infected with a virus capable of causing Viral Hemorrhagic Fever by administering a COX-2 selective NSAID. In block 1, a patient becomes infected (e.g., via a bite from a mosquito, tick, contact with an infected person) with a virus. In block 2, depending on the severity of the symptoms, the patient may choose to do nothing if asymptomatic, self-treat if experiencing an infection of mild severity, or contact a healthcare provider seeking medical treatment if the symptoms are more severe or the patient is more physically compromised (e.g., elderly, child). In block 3, the patient is treated with a course of therapy with a COX-2 selective NSAID to address symptoms of the suspected VHF, either via self-administration, if direct access to the COX-2 selective NSAID by the patient is possible, or as directed by a healthcare provider. In block 4, the healthcare provider assesses the patient for symptoms to determine if a VHF is likely involved, and additionally may employ more sophisticated diagnostic techniques (e.g., identification of the viral pathogen via anti-body test or other methods, quantitative viral load assessment). In block 6, with a confirmed diagnosis of a VHF, the healthcare provider can choose to treat the symptoms only of the infection with a COX-2 selective NSAID, or choose to treat to suppress viral replication. In block 7, the healthcare provider utilizes a dose and duration of treatment with a COX-2 selective NSAID designed to suppress viral replication of the now identified pathogen. In block 8, after a course of treatment to suppress viral replication, the healthcare provider reassesses their treatment results and can pursue another course of therapy to suppress viral replication, treat for symptom relief only, or discontinue treatment with a COX-2 selective NSAID. In block 9, the healthcare provider, or self-treating patient, who is treating for symptom relief reassesses their treatment results and can modify the dose or duration of treatment with the COX-2 selective NSAID, add acetaminophen if additional fever reduction or pain relief is necessary and the dose limit of the COX-2 selective NSAID has been reached, self-refer to a healthcare provider if symptoms persist or worsen (in the case of a self-treating patient) or discontinue treatment with a COX-2 selective NSAID if conditions warrant.

### EXAMPLES

### Example 1

Rofecoxib is a COX-2 selective NSAID with potent anti-pyretic and anti-inflammatory properties. Rofecoxib is highly COX-2 selective, with a 35-fold COX-2 versus COX-1 selectivity, In some embodiments, the treatment with rofecoxib can be as described in U.S. application Ser. No. 16/716,242 titled “Purified Forms of Rofecoxib, Methods of Manufacture and Use”, U.S. Provisional Application No. 62/934,898 titled “Novel Dosage of Rofecoxib and Related Methods”, U.S. Provisional Application No. 62/945,641 titled “Aqueous Formulations of Water Insoluble COX-2 Inhibitors.”

Unlike non-selective NSAIDs, rofecoxib, at doses up to 375 mg/day, has demonstrated to not affect platelet aggregation or bleeding time. Rofecoxib has demonstrated significant improvements in GI tolerability and safety events versus non-selective NSAIDs. Rofecoxib was withdrawn from the market worldwide in 2004 due to evidence of a CV safety issue and has never been returned to the market. Rofecoxib is an oral once daily product with a half-live of approximately 17 hours. Rofecoxib has been shown to have rapid and sustained anti-inflammatory efficacy, with a median onset of action in acute pain settings of 45 minutes and sustained efficacy over 24 hours with a single dose.

Detailed Exemplary Embodiments

A detailed exemplary embodiment of the present disclosure could include a method of treating fever, pain, and inflammation in a patient infected with a virus capable of causing Viral Hemorrhagic Fever, comprising: administering to the patient a COX-2 selective NSAID at a sufficient dose and duration such that the patient experiences a reduction in the symptoms of fever, pain, and inflammation. In some embodiments, the COX-2 selective NSAID is administered orally. In some embodiments, the COX-2 selective NSAID is administered parenterally. In some embodiments, the COX-2 selective NSAID is administered at least once daily for one day, 3 days, 5 days, 7 days, 10 days, or 14 days. In some embodiments, the sufficient dose of the COX-2 selective NSAID is 12.5 mg/day to 375 mg/day. In some embodiments, the COX-2 selective NSAID is rofecoxib and the sufficient dose is 12.5 mg/day to 50 mg/day of rofecoxib.

A further potential embodiment of the present disclosure could include a method of treating fever, pain, and inflammation in a patient infected with a virus capable of causing Viral Hemorrhagic Fever comprising administering a COX-2 selective NSAID to the patient, wherein the virus capable of causing Viral Hemorrhagic Fever is the Dengue Virus.

A further potential embodiment of the present disclosure could include a method of treating fever, pain, and inflammation in a patient infected with a virus capable of causing Viral Hemorrhagic Fever comprising administering a COX-2 selective NSAID to the patient, wherein the COX-2 selective NSAID is selected from the group consisting of celecoxib, rofecoxib, valdecoxib, parecoxib, or lumiracoxib. In some embodiments, the COX-2 selective NSAID is administered at least once daily for one day, 3 days, 5 days, 7 days, 10 days, or 14 days.

A further potential embodiment of the present disclosure could include a method of treating fever, pain, and inflammation in a patient infected with a virus capable of causing Viral Hemorrhagic Fever comprising administering a COX-2 selective NSAID to the patient, wherein the COX-2 selective NSAID is administered as an initial loading dose during the febrile phase of the viral infection, followed by a lower maintenance dose for the remainder of the time course of symptoms.

A further potential embodiment of the present disclosure could include a method of treating fever, pain, and inflammation in a patient infected with a virus capable of causing Viral Hemorrhagic Fever comprising administering a COX-2 selective NSAID to the patient, wherein the COX-2 selective NSAID rofecoxib is administered as an initial loading dose of between 25 mg to 375 mg/day, followed by a maintenance dose of between 12.5 mg to 50 mg/day. In some embodiments, the maintenance dose is administered for at least one day. In some embodiments, the maintenance dose is administered for about 3 days, 5 days, 7 days, 10 days, or 14 days.

A detailed exemplary embodiment of the present disclosure could include a method of treating a Viral Hemorrhagic Fever patient infected with a virus capable of causing Viral Hemorrhagic Fever, comprising: administering to the patient a COX-2 selective NSAID at a sufficient dose to cause a reduction in the viral level of said virus.

A further potential embodiment of the present disclosure could include a method of treating a Viral Hemorrhagic Fever patient infected with a virus capable of causing Viral Hemorrhagic Fever comprising administering a COX-2 selective NSAID to the patient, wherein the COX-2 selective NSAID is selected from the group consisting of celecoxib, rofecoxib, valdecoxib, parecoxib, or lumiracoxib.

A further potential embodiment of the present disclosure could include a method of treating fever, pain, and inflammation in a patient infected with a virus capable of causing Viral Hemorrhagic Fever comprising administering a COX-2 selective NSAID to the patient in combination with acetaminophen.

A further potential embodiment of the present disclosure could include a method of treating fever, pain, and inflammation in a patient infected with a virus capable of causing Viral Hemorrhagic Fever comprising administering a COX-2 selective NSAID to the patient, wherein the COX-2 selective NSAID is provided in quantity sufficient for a single course of therapy.

A further potential embodiment of the present disclosure could include a method of treating a Viral Hemorrhagic Fever patient infected with a virus capable of causing Viral Hemorrhagic Fever comprising administering a COX-2 selective NSAID to the patient, wherein the COX-2 selective NSAID rofecoxib is administered as an initial loading dose of between 25 mg/day and 375 mg/day, followed by a maintenance dose between 12.5 mg/day and 50 mg/day. In some embodiments, the maintenance dose is administered for at least one day. In some embodiments, the maintenance dose is administered for about 3 days, 5 days, 7 days, 10 days, or 14 days.

A detailed exemplary embodiment of the present disclosure could include a method of treating fever, pain, and inflammation in a patient infected with a virus exhibiting symptoms of the Zika Virus, comprising: administering to the patient a COX-2 selective NSAID such that the patient experiences a reduction in the symptoms of fever, pain, and inflammation. In some embodiments, the COX-2 selective NSAID is administered orally.

A detailed exemplary embodiment of the present disclosure could include a method of treating a patient infected with the Zika Virus, comprising: administering to the patient a COX-2 selective NSAID at a sufficient dose such that the patient experiences a reduction in viral load of the Zika virus. In some embodiments, the COX-2 selective NSAID is administered orally.

### REFERENCES

The following related art documents are incorporated by reference in their entirety.

### OTHER PUBLICATIONS

European Medical Agency, Arcoxia 30 60 90 120 mg film-coated tablets—Summary of Product Characteristics (SPC)-(eMC), Last Updated on eMC 25 Jul. 2016
Australian Register of Therapeutic Goods, ARCOXIA Tablet PI (MK0663-AUS-2015-011356), Last Updated Jul. 14, 2017.
Diaz-Quijano et al. Predictors of spontaneous bleeding in patient with acute febrile syndrome from a dengue endemic area J Clin Virology 49 (2010) 11-15
US Food and Drug Administration. VIOXX (Rofecoxib) U.S. Prescribing Information May 9, 2016.
Epidemiology Unit, Ministry of Health, Sri Lanka. Advice for Dengue patients who are on home based care temporarily, 2017 (http://www.epid.gov.1k/web/images/pdf/DHF/Leaflet/dengue leaflet english.pdf)
United Nations, Caring-for-a-Dengue-patient, 2017 (http://lk.one.un.org/wp-content/uploads/2017/07/Caring-for-a-Dengue-patient.pdf)
World Health Organization, Dengue Fact Sheet, http://www.who.int/mediacentre/factsheets/fs117/en/
Sameh Elawdy, et al. Association between severity of dengue infection and dengue viral load: a systematic review and meta-analysis. PROSPERO 2016 CRD42016039864 http://www.crd.york.ac.uk/PROSPERO/display record.php?ID=CRD42016039864
Coxib and traditional NSAID Trialists' (CNT) Collaboration. Vascular and upper gastrointestinal effects of non-steroidal anti-inflammatory drugs: meta-analyses of individual participant data from randomised trials. The Lancet 2013; 382: 769-79.
McGettigan P, Henry D. Cardiovascular risk with non-steroidal anti-inflammatory drugs: systematic review of population-based controlled observational studies. PLoS Med 2011; 8: e1001098.

### Note on Scope of the Present Disclosure

The above are descriptions and preferred embodiments are examples only and are in no way intended to limit the potential combinations under which the present disclosure could be utilized.

What is claimed is:

1. A method for treating one or more symptoms in a subject having Viral Hemorrhagic Fever, the method comprising administering a therapeutically effective amount of a COX-2 selective Non-Steroidal Anti-Inflammatory Drug (NSAID) or a pharmaceutically acceptable salt thereof.

2. The method of claim 1, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

3. The method of claim 1, wherein the Viral Hemorrhagic Fever is caused by one or more RNA viruses derived from a family of viruses selected from Arenaviridae, Bunyaviridae, Filoviridae, Flaviviridae, and any combinations thereof.

4. The method of claim 3, wherein the Viral Hemorrhagic Fever is caused by one or more RNA viruses from the Flaviviridae family of viruses.

5. The method of claim 4, wherein the Viral Hemorrhagic Fever is caused by a Dengue Virus.

6. The method of claim 1, wherein the therapeutically effective amount of the COX-2 selective NSAID causes a reduction in viral load.

7. The method of claim 1, wherein the COX-2 selective NSAID is administered orally.

8. The method of claim 7, wherein the COX-2 selective NSAID is administered at a duration and a dosage such that the subject experiences a reduction in the one or more symptoms.

9. The method of claim 8, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

10. The method of claim 1, wherein the therapeutically effective amount of the COX-2 selective NSAID is provided in a quantity sufficient for a single course of therapy.

11. The method of claim 10, wherein the therapeutically effective amount of the COX-2 selective NSAID is provided in a single course of therapy package.

12. The method of claim 1, wherein the therapeutically effective amount of the COX-2 selective NSAID is at a dose matched to the time course of the Viral Hemorrhagic Fever.

13. The method of claim 12, wherein the dose provides maximum anti-pyretic effect during the febrile phase of an infection.

14. The method of claim 13, further comprising administering a lower maintenance dose during the critical phase and/or recovery phase of the infection compared to the dose matched to the time course of the Viral Hemorrhagic Fever.

15. The method of claim 1, wherein the COX-2 selective NSAID is administered as a loading dose during a febrile stage of a viral infection, and a lower maintenance dose during the critical phase and/or recovery phase of the infection.

16. The method of claim 1, wherein the COX-2 selective NSAID is administered at a dose that does not affect platelet aggregation or bleeding time.

17. The method of claim 1, wherein the COX-2 selective NSAID is selected from celecoxib, rofecoxib, valdecoxib, parecoxib, lumiracoxib, and any combinations thereof.

18. The method of claim 17, wherein the COX-2 selective NSAID is rofecoxib.

19. The method of claim 18, wherein the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg.

20. The method of claim 18, wherein the rofecoxib is administered at a dose of about 12.5 mg/day, about 15 mg/day, about 20 mg/day, about 25 mg/day, about 30 mg/day, about 40 mg/day, or about 50 mg/day.

21. The method of claim 18, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

22. The method of claim 21, further comprising administering a maintenance dose of rofecoxib of about 12.5 mg/day, about 20 mg/day, about 25 mg/day, about 30 mg/day, about 40 mg/day, or about 50 mg/day.

23. The method of claim 22, wherein the maintenance dose of rofecoxib is administered for up to 3 days, up to 5 days, up to 7 days, up to 10 days, or up to 14 days.

24. The method of claim 1, wherein the COX-2 selective NSAID is administered in combination with acetaminophen.

25. The method of claim 24, wherein the COX-2 selective NSAID is selected from celecoxib, rofecoxib, valdecoxib, parecoxib, lumiracoxib, and combinations thereof.

26. The method of claim 25, wherein the COX-2 selective NSAID is rofecoxib.

27. The method of claim 26, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

28. The method of claim 27, further comprising administering a maintenance dose of rofecoxib of about 12.5 mg/day, about 15 mg/day, about 20 mg/day, about 25 mg/day, about 30 mg/day, about 40 mg/day, or about 50 mg/day.

29. The method of claim 28, wherein the maintenance dose of rofecoxib is administered for up to 3 days, up to 5 days, up to 7 days, up to 10 days, or up to 14 days.

30. A method for treating a subject infected with a virus capable of causing Viral Hemorrhagic Fever, the method comprising:
assessing the subject for one or more symptoms of Viral Hemorrhagic Fever, and if one or more symptoms of Viral Hemorrhagic Fever is present, administering a therapeutically effective amount of a COX-2 selective Non-Steroidal Anti-Inflammatory Drug (NSAID) or a pharmaceutically acceptable salt thereof to the subject.

31. The method of claim 30, further comprising:
reassessing symptoms following the administration of the therapeutically effective amount of the COX-2 selective NSAID; and
adjusting treatment according to the reassessed symptoms, wherein the adjusting comprises administering further COX-2 selective NSAID if the reassessed symptoms are worse, not improved or improved but not gone compared to the assessed symptoms and terminating administering COX-2 selective NSAID if the reassessed symptoms are gone compared to the assed symptoms.

32. The method of claim 30, further comprising identifying the virus capable of causing Viral Hemorrhagic Fever in the infected subject.

33. The method of claim 30, wherein the therapeutically effective amount of the COX-2 selective NSAID comprises a dose and duration of treatment sufficient to suppress viral replication of the identified viral pathogen or decrease viral load.

34. The method of claim 30, wherein the COX-2 selective NSAID is selected from celecoxib, rofecoxib, valdecoxib, parecoxib, lumiracoxib, and combinations thereof.

35. The method of claim 34, wherein the COX-2 selective NSAID is rofecoxib.

36. The method of claim 35, wherein the rofecoxib is administered at a dose of about 12.5 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

37. The method of claim 35, wherein the rofecoxib is administered at a dose of about 12.5 mg/day, about 20 mg/day, about 25 mg/day, about 30 mg/day, about 40 mg/day, or about 50 mg/day.

38. The method of claim 35, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

39. The method of claim 38, further comprising administering a maintenance dose of rofecoxib of about 12.5 mg/day, about 20 mg/day, about 25 mg/day, about 30 mg/day, about 40 mg/day, or about 50 mg/day.

40. The method of claim 39, wherein the maintenance dose of rofecoxib is administered for up to 3 days, up to 5 days, up to 7 days, up to 10 days, or up to 14 days.

41. A method for treating a subject infected with a virus capable of causing Viral Hemorrhagic Fever, the method comprising:
administering to the subject a COX-2 selective Non-Steroidal Anti-Inflammatory Drug (NSAID) or a pharmaceutically acceptable salt thereof at a sufficient dose to cause a reduction in viral load of the virus.

42. The method of claim 41, wherein the Viral Hemorrhagic Fever is caused by one or more RNA viruses derived from a family of viruses selected from Arenaviridae, Bunyaviridae, Filoviridae, Flaviviridae, and any combinations thereof.

43. The method of claim 42, wherein the Viral Hemorrhagic Fever is caused by one or more RNA viruses from the Flaviviridae family of viruses.

44. The method of claim 43, wherein the Viral Hemorrhagic Fever is caused by a Dengue Virus.

45. The method of claim 41, wherein the COX-2 selective NSAID is selected from celecoxib, rofecoxib, valdecoxib, parecoxib, lumiracoxib, and combinations thereof.

46. The method of claim 45, wherein the COX-2 selective NSAID is rofecoxib.

47. The method of claim 46, wherein the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

48. The method of claim 46, wherein the rofecoxib is administered at a dose of about 12.5 mg/day, about 20 mg/day, about 25 mg/day, about 30 mg/day, about 40 mg/day, or about 50 mg/day.

49. The method of claim 46, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

50. The method of claim 49, further comprising administering a maintenance dose of rofecoxib of about 12.5 mg/day, about 20 mg/day, about 25 mg/day, about 30 mg/day, about 40 mg/day, or about 50 mg/day.

51. The method of claim 50, wherein the maintenance dose of rofecoxib is administered for up to 3 days, up to 5 days, up to 7 days, up to 10 days, or up to 14 days.

52. The method of claim 46, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day, followed by a maintenance dose of about 12.5 mg/day, about 20 mg/day, about 25 mg/day, about 30 mg/day, about 40 mg/day, or about 50 mg/day.

53. A method for treating one or more symptoms in a subject having symptoms of Zika Virus infection, the method comprising:
administering to the subject a COX-2 selective Non-Steroidal Anti-Inflammatory Drug (NSAID) or a pharmaceutically acceptable salt thereof such that the subject experiences a reduction in the one or more symptoms.

54. The method of claim 53, wherein the COX-2 selective NSAID is administered orally.

55. The method of claim 53, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

56. The method of claim 53, wherein the COX-2 selective NSAID is selected from celecoxib, rofecoxib, valdecoxib, parecoxib, lumiracoxib, and combinations thereof.

57. The method of claim 56, wherein the COX-2 selective NSAID is rofecoxib.

58. The method of claim 57, wherein the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

59. The method of claim 57, wherein the rofecoxib is administered at a dose of about 12.5 mg/day, about 20 mg/day, about 25 mg/day, about 30 mg/day, about 40 mg/day, or about 50 mg/day.

60. The method of claim 57, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

61. The method of claim 60, further comprising administering a maintenance dose of rofecoxib of about 12.5 mg/day, about 20 mg/day, about 25 mg/day, about 30 mg/day, about 40 mg/day, or about 50 mg/day.

62. The method of claim 61, wherein the maintenance dose of rofecoxib is administered for up to 3 days, up to 5 days, up to 7 days, up to 10 days, or up to 14 days.

63. A method for treating a subject infected with Zika Virus, the method comprising:
administering to the subject a COX-2 selective NSAID or a pharmaceutically acceptable salt thereof at a sufficient dose such that the subject experiences a reduction in viral load of the Zika virus.

64. The method of claim 63, wherein the COX-2 selective NSAID is administered orally.

65. The method of claim 63, wherein the COX-2 selective NSAID is selected from celecoxib, rofecoxib, valdecoxib, parecoxib, lumiracoxib, and combinations thereof.

66. The method of claim 65, wherein the COX-2 selective NSAID is rofecoxib.

67. The method of claim 66, wherein the rofecoxib is administered at a dose of about 12.5 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

68. The method of claim 66, wherein the rofecoxib is administered at a dose of about 12.5 mg/day, about 20 mg/day, about 25 mg/day, about 30 mg/day, about 40 mg/day, or about 50 mg/day.

69. The method of claim 68, wherein the rofecoxib is administered as an initial loading dose of about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, or about 375 mg/day.

70. The method of claim 69, further comprising administering a maintenance dose of rofecoxib of about 12.5 mg/day, about 20 mg/day, about 25 mg/day, about 30 mg/day, about 40 mg/day, or about 50 mg/day.

71. The method of claim 70, wherein the maintenance dose of rofecoxib is administered for up to 3 days, up to 5 days, up to 7 days, up to 10 days, or up to 14 days.
